(19) United States
(12) Patent Application Publication
(10) Pub. No.: US 2006/0286131 A1
(43) Pub. Date: Dec. 21, 2006

(54) Use of stellate ganglion block for the treatment of hot flashes and other conditions

(76) Inventor: Eugene G. Lipov, Chicago, IL (US)

Correspondence Address: BRIAN J. LUM;ICE MILLER LLP, SUITE 3500, 200 WEST MADISON STREET, CHICAGO, IL, 60606, US

(21) Appl. No.: 11/376,533

(22) Filed: Mar. 15, 2006

Related U.S. Application Data
(60) Provisional application No. 60/691,055, filed on Jun. 16, 2005.
(60) Provisional application No. 60/697,233, filed on Jul. 7, 2005.

(51) Int. Cl. A61K 9/00

(52) U.S. Cl. 424/400

(57) ABSTRACT

The present invention is directed to a method for the treatment of a patient suffering from hot flashes and other symptoms comprising the step of administering a stellate ganglion block to the patient to alleviate the symptoms. The stellate ganglion block may be followed by a sympathectomy to provide permanent relief.

This application claims priority to U.S. Provisional Application No. 60/691,055, filed Jun. 16, 2005, and U.S. Provisional Application No. 60/697,233, filed Jul. 7, 2005, herein incorporated by reference.

### BACKGROUND OF THE INVENTION

The present invention is directed to methods and kits for the treatment of hot flashes other symptoms of menopause, and other conditions.

Hot flashes are experienced by many women undergoing menopause. A hot flash is a feeling of warmth, sometimes associated with flushing, that spreads over the body and may be accompanied by perspiration. Hot flashes may vary in severity and may last for a short period of time in some women, but can last for a decade or more in other women. Although the cause of hot flashes is not completely understood, hot flashes may be related to fluctuations of hormone levels experienced during menopause. Hot flashes also may be experienced secondary to mastectomy and other cancer-related treatments, particularly cancer treatments that affect hormone levels.

Traditionally, hot flashes have been treated with hormone therapy. Hormone replacement medications (usually estrogen or a combination of estrogen and progesterone) are effective in reducing the frequency of hot flashes and their severity. Generally, these medications decrease the frequency of hot flashes by about 80 to 90%. However, hormone replacement therapy is associated with increased risk of heart attack, stroke, blood clots, and breast cancer. Thus, alternative treatments are desirable.

A stellate ganglion block is an injection of local anesthetic into the stellate ganglion nerves—nerve tissue located in the neck on either side of the larynx. The injection of local anesthetic into these nerves blocks impulses, which may in turn reduce pain, swelling, color, and sweating changes in the upper extremity and may improve mobility. Stellate ganglion blocks are known as part of the treatment of Reflex Sympathetic Dystrophy (RSD), Sympathetic Maintained Pain, Complex Regional Pain Syndrome (CRPS), and Herpes Zoster (shingles) involving the head and face and/or upper extremities.

It has been found that the stellate ganglion block can be used to treat hot flashes. In an initial study, complete asymptomatic relief of two to five weeks has been experienced with a single stellate ganglion block, with increasingly longer periods of relief following subsequent blocks.

### SUMMARY OF THE INVENTION

The present invention is directed to a method for the treatment of a patient suffering from a menopause-related condition comprising the step of administering a stellate ganglion block to provide relief from the menopause-related condition. Illustratively, the patient is suffering from hot flashes and the stellate ganglion block alleviates the symptoms of hot flashes.

Additional features of the present invention will become apparent to those skilled in the art upon consideration of the following detailed description of preferred embodiments exemplifying the best mode of carrying out the invention as presently perceived.

### BRIEF DESCRIPTION OF THE DRAWINGS

FIG. 1 is a perspective view showing various vertebrae and nerves in the neck and needle placement for a stellate ganglion block.

### DETAILED DESCRIPTION

A ganglion is a group of nerve cell bodies. The stellate ganglion is a ganglion located in the neck, formed by the fusion of the inferior cervical and the first thoracic ganglions. These ganglions meet anterior to the C7 vertebra.

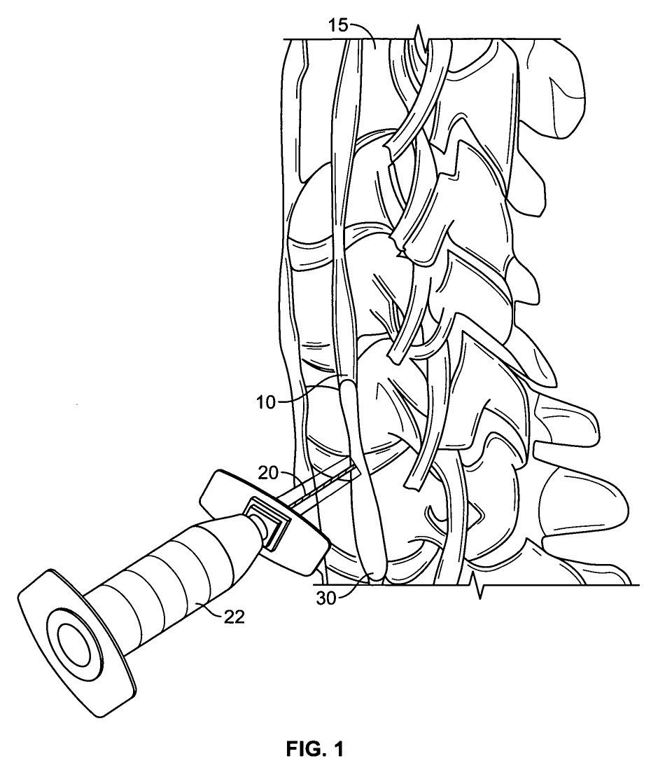

A stellate ganglion block is illustratively performed by placing the patient in the supine position (on the back) with the neck slightly extended and the jaw open. While the patient is usually conscious for the procedure, a medication such as Versed (midazolam hydrochloride), fentanyl, or other sedative is often administered, to relax the patient. A local anesthetic is also usually administered, to numb the skin and tissue down to the ganglion nerve. A needle is inserted toward the stellate ganglion. FIG. 1 shows the stellate ganglion 10 extending anterior to vertebral column 15. The needle 20 is shown inserted adjacent to the stellate ganglion 10. In the illustrative block, a contrast solution is injected through needle 20 via syringe 22, and the needle position is confirmed by fluoroscopy or other means, as are known in the art.

The anesthetic 30 is then injected via needle 20 into stellate ganglion 10. Illustratively, 5 cc 0.375% Marcaine® (bupivacaine) may be used. However, it is understood that other anesthetics, illustratively lidocaine, morphine or other opioids, clonidine, botox, or tetracine, or a combination of anesthetic and epinephrine, may be used and that the dosage range may vary depending on the particular anesthetic and other factors, as are known in the art. Needle 20 is removed, and the patient is optionally placed in the sitting position to facilitate the spread of anesthesia inferiorly to the stellate ganglion. Illustratively, longer acting anesthetics such as bupivacaine are used, but other anesthetics may be used, as are known in the art. The stellate ganglion block may also be performed by radiofrequency lesioning, as is known in the art.

While fluoroscopy is used in the illustrative method, stellate ganglion blocks have been traditionally performed blindly by palpating the transverse process of C6 and infiltrating a large volume (as much as 20 mL) of local anesthetic. It is understood that this technique is dependent on enough volume reaching the stellate ganglion to result in an effective block and requires a larger volume of anesthetic. While such techniques may be used to treat hot flashes, it is understood that image-guided stellate ganglion blocks have the advantages of increased safety and accuracy compared with blind injections because the needle can be accurately placed close to the ganglion itself. Because of the more accurate placement of the needle to the ganglion, a smaller amount of local anesthetic can be used, resulting in a decrease in adverse effects.

Patients may experience side effects such as nasal congestion, a hoarse voice, and a warm tingling sensation in the arm and hand, and may display symptoms associated with “Horner's syndrome” such as a bloodshot, droopy eye on the side of the injection. These side effects usually disappear after several hours. If the first injection reduces or eliminates hot flashes for a period of time, subsequent blocks may be given. It has been found that the duration of relief often increases with each subsequent injection. Surgical sympathectomy may be performed for permanent relief from hot flashes. Sympathectomies are currently performed on patients who suffer from excessive perspiration and the surgical techniques are known in the art. Generally, a sympathectomy would be performed if a patient responds well to the stellate ganglion block, to provide permanent relief from severe hot flashes.

In addition to hot flashes, it is believed that stellate ganglion blocks may aid in the treatment and/or prevention of other conditions associated with menopause, including osteoporosis and fat deposits. For ongoing medical conditions such as osteoporosis, a series of stellate ganglion blocks may be administered periodically, for months or years. The blocks may be administered every week or two to every several months. The blocks may be administered on a regular periodic basis, or the length of time between blocks may change, illustratively increasing. The stellate ganglion block may also prove to be an effective treatment for overweight patients, resulting in weight loss and/or prevention or treatment of diabetes mellitus. A series of additional stellate ganglion blocks may be administered periodically, until a desired weight is attained or to maintain a desired weight. Finally, the stellate ganglion block may be effective for mood disorders, schizophrenia, and other psychiatric disorders, with repeat treatments as necessary. A sympathectomy may also be effective for these conditions.

Melatonin is a hormone produced by the pineal gland and is involved in synchronizing the body's hormone secretions and setting the brain's internal clock. Typically, the body's production of melatonin begins around dusk, increases during the night, and then decreases as daylight appears. Melatonin supplements are widely used to relieve sleep disorders, including insomnia and the symptoms of jet lag. Melatonin is also used to treat mood disorders and depression, illustratively seasonal affective disorder.

In another embodiment of the present invention, the stellate ganglion block may be used along with melatonin to treat sleep disorders, mood disorders, and depression. In one illustrative embodiment, the stellate ganglion block is performed contemporaneously with administration of melatonin. The melatonin may be administered prior to or subsequent to the stellate ganglion block, illustratively within an hour of the stellate ganglion block, but it is understood that other time periods between the administration of melatonin and stellate ganglion block are within the scope of this invention. Melatonin is often administered orally, but other dosage forms are known and may be used within the scope of this invention. Illustratively, this treatment is performed in the morning, when melatonin levels are lowest. However, this treatment may be effective at other times of the day.

### EXAMPLE I

Six women suffering from severe menopausal hot flashes each received a standard stellate ganglion block (0.375% Marcaine, 5cc), as described above. Hot flashes were assessed by self-reporting pre and post stellate ganglion block.

The initial stellate ganglion block was successful in all six subjects, as evidenced by a positive Horner's syndrome and anhydrosis. Successful stellate ganglion block resulted in complete alleviation of hot flashes for times ranging from 2-5 weeks. Patients returned for a follow-up stellate ganglion block after mild hot flashes returned. A second stellate ganglion block produced additional asymptomatic periods of relief ranging from 4-18 weeks. In each case, repeated blocks provided hot flash relief equal to or greater than that of the initial block. Two patients who submitted for a third stellate ganglion block reported 15 and 48 weeks relief.

Accordingly, stellate ganglion blocks appear to be related to relief of hot flashes. Repeat stellate ganglion block results in efficacious multiple week relief of severe hot flashes associated with menopause.

Although the invention has been described in detail with reference to preferred embodiments, variations and modifications exist within the scope and spirit of the invention as described and defined in the following claims.

1. A method for the treatment of a patient suffering from hot flashes comprising the step of
administering a stellate ganglion block to the patient to alleviate the symptoms of hot flashes.

2. The method of claim 1 wherein the stellate ganglion block comprises administering an anesthetic to the stellate ganglion.

3. The method of claim 1 wherein the anesthetic is bupivacaine.

4. The method of claim 1 wherein the stellate ganglion block provides asymptomatic relief from hot flashes for at least about 2 weeks.

5. The method of claim 1 further comprising the step of
administering a second stellate ganglion block.

6. The method of claim 5 wherein the second stellate ganglion block is administered subsequent to the return of hot flash symptoms.

7. The method of claim 5 wherein the second stellate ganglion block is administered at least two weeks subsequent to the stellate ganglion block.

8. The method of claim 1 further comprising the step of
administering a series of additional stellate ganglion blocks, each additional stellate ganglion block administered subsequent to a return of hot flash symptoms.

9. The method of claim 8 wherein the time period for the return of the hot flash symptoms increases following each subsequent ganglion block.

10. A method for the treatment of a patient suffering from a menopause-related condition comprising the step of
administering a stellate ganglion block to provide relief from the menopause-related condition.

11. The method of claim 10 wherein the menopause-related condition is selected from the group consisting of hot flashes, osteoporosis, fat deposits, and a combination thereof.

12. The method of claim 1 wherein the stellate ganglion block is administered subsequent to a cancer treatment performed on the patient.

13. The method of claim 12 wherein the cancer treatment is a mastectomy.

14. The method of claim 1 wherein the patient is female.

15. The method of claim 1 further comprising the step of performing a sympathectomy if the patient obtains relief subsequent to the stellate ganglion block.

16. A method for the treatment of an overweight patient comprising the step of
administering a stellate ganglion block to the patient to achieve a more desirable weight or to prevent development of diabetes mellitus.

17. The method of claim 16 further comprising the step of
administering a series of additional stellate ganglion blocks, each after a set time period, until a desired weight is achieved.

18. A method for the treatment of a patient suffering from mood disorders or schizophrenia comprising the step of
administering a stellate ganglion block to the patient to alleviate symptoms of mood disorders or schizophrenia.

19. The method of claim 18 further comprising the step of performing a sympathectomy.

20. A method of treatment of a patient wherein the treatment comprising the steps of
administering a stellate ganglion block to the patient, and
administering melatonin to the patient.

21. The method of claim 20 wherein the stellate ganglion block and the melatonin are administered within one hour of each other.

22. The method of claim 20 wherein the patient is suffering from a condition selected from the group consisting of a sleep disorder, a mood disorders, and depression, and the treatment alleviates symptoms of the condition.
